(19) United States
(12) Patent Application Publication
(10) Pub. No.: US 2015/0151135 A1
(43) Pub. Date: Jun. 4, 2015

(54) TRANSDERMAL DELIVERY OF DNA VACCINES USING NON-THERMAL PLASMA

(71) Applicants: Sameer Kalghatgi, Copley, OH (US); Daphne Pappas Antonakas, Hudson, OH (US); Tsung-Chan Tsai, Cuyahoga Falls, OH (US); Robert L. Gray, Hudson, OH (US)

(72) Inventors: Sameer Kalghatgi, Copley, OH (US); Daphne Pappas Antonakas, Hudson, OH (US); Tsung-Chan Tsai, Cuyahoga Falls, OH (US); Robert L. Gray, Hudson, OH (US)

(73) Assignee: EP TECHNOLOGIES LLC, Akron, OH (US)

(21) Appl. No.: 14/560,343

(22) Filed: Dec. 4, 2014

Related U.S. Application Data
(60) Provisional application No. 61/911,536, filed on Dec. 4, 2013.

(51) Int. Cl. A61N 1/44; A61N 5/06; A61K 39/00; A61M 37/00

(52) U.S. Cl. CPC A61N 1/44; A61M 37/00; A61N 5/0616; A61K 39/00; A61M 2037/0007; A61K 2039/53; A61K 48/00

(57) ABSTRACT

Exemplary systems and methods of delivering DNA vaccines are disclosed herein. An exemplary methodology of delivering DNA vaccines includes providing a plasma generator for applying plasma to a treatment area for a sufficient period of time to open one or more pores. Applying a topical DNA vaccine to the treatment area and waiting for a period of time to allow the DNA vaccine to travel through the one or more pores. The exemplary methodology further includes applying plasma to the treatment area at a setting sufficient to cause intracellular uptake of the DNA vaccine.

### RELATED APPLICATIONS

This application claims priority to and the benefits of U.S. Provisional Patent Application Ser. No. 61/911,536 filed on Dec. 4, 2013 and entitled “TRANSDERMAL DELIVERY OF DNA VACCINES USING NON-THERMAL PLASMA,” which is incorporated herein by reference in its entirety.

### TECHNICAL FIELD

The present invention relates generally to delivery of DNA vaccines and more particularly to delivering DNA vaccines using non-thermal plasma (cold plasma) for intercellular delivery of DNA vaccines across skin, tissue or tumor and/or to promote intracellular uptake of DNA vaccines. Tissue refers to epithelial, mucosal, connective and muscle tissue in the body.

### BACKGROUND

Vaccines are one of the most important discoveries of modern medicine and the most beneficial treatment a physician can provide to a patient. Yet a number of vaccine preventable diseases await the technology to elicit the appropriate protective or therapeutic immune response. Most vaccines elicit antibody responses, however, cell mediated immune responses, including CD8 T cells are necessary to prevent, control or treat intracellular bacterial, fungal and viral diseases as well as chronic diseases, including diabetes, cancer, etc. and deadly diseases like Ebola.

DNA vaccination is advantageous because it does not integrate into the host DNA, it is cost effective to produce and easily stored, it can be highly specific for tissue and/or cell type and can be made to vaccinate against multiple agents simultaneously. The skin is an ideal target for DNA vaccination due to the large surface area and the presence of antigen presenting cells like Langerhans's and dermal dendritic cells, specialized for induction of immunity

DNA vaccines can cause both cell mediated immune responses and antibody responses. Accordingly, DNA vaccines represent an attractive alternative to other modes of vaccination. DNA vaccines consist of a plasmid (circle of DNA) that contains genes for the immunogenic proteins necessary to elicit protection, genes for proteins to enhance the immune response, and DNA sequences necessary for transcription into RNA, translation into protein in mammalian cells, and amplification of the plasmid in bacterial but not mammalian cells. Immune responses to DNA vaccines resemble the response to a viral infection, but are safer since DNA does not spread or cause disease. DNA is also relatively easy to manufacture and stable to the environmental proteases and nucleases. DNA vaccines may be used to generate the immune responses necessary to prevent or treat diseases, such as, for example, HSV, AIDS, hepatitis C, cancer, Ebola and the like that have eluded vaccine development by more conventional means.

A roadblock to acceptance of DNA vaccines for prophylactic or therapeutic vaccination is difficulty in promoting efficient delivery and cellular uptake and appropriate cell mediated immune response. Either due to low expression or lack of immune recognition, injection of plasmid DNA alone does not elicit a strong enough immune response for protective vaccination. Several methods for delivery and uptake of DNA vaccines including lipid-mediated delivery, jet injections, gene guns and sonoporation, have been tested without much success.

Recent developments with the genetics of DNA vaccines and the use of electroporation for in vivo delivery of DNA vaccines have increased efficiency of expression to levels that are practical. Electroporation uses pulsed electric currents to open pores in cell membranes (a process called permeabilization) and allows the intradermally injected DNA to be taken up by skin cells and immune cells residing in the skin. Electroporation requires DNA injection into the skin or muscle, direct electrode contact with skin or insertion of electrodes in to muscle and application of direct current to promote cellular uptake of DNA.

Electroporation as a drug delivery method has several drawbacks including pain, muscle contractions upon application and also leads to current induced tissue damage. These drawbacks have limited its widespread adoption. Indeed, the pain associated with electroporation is severe enough that it is unlikely that doctors or care providers would recommended its use in children or elderly. In addition, electroporation can only be used on an area that is between about 5 mm² to about 7 mm².

One study by Richard J Connolly, from the University of South Florida and titled Plasma Mediated Molecular Delivery showed that the non-thermal plasma can also deliver pulsed electric fields to the skin and demonstrated that this method can safely promote cellular uptake of intradermally injected DNA vaccines. The experiments injected 100 μg of JRFLgp120 plasmid intradermally in a volume of 50 μl. The bolus was exposed to positive or negative polarity plasma for 10 minutes. Like electroporation, this method requires DNA injection into the skin with needles, an application method that is painful and results in the production of biohazardous waste that must be carefully disposed of. Also, this proposed method involves the use of expensive noble gases like helium to generate plasma.

Transdermal, needle-free delivery of vaccines is desirable for certain groups of individuals that cannot tolerate the pain of an injection, such as children or seniors. Intranasal delivery has attracted a lot of interest, but the limitations of the total vaccine volume that can be delivered are a challenge. The delivered vaccines become diluted in mucosal secretions, attacked by proteases and nucleases, and excluded by epithelial barriers. So, relatively large doses of vaccine are required and it is impossible to determine exactly what dose actually crosses the mucosa. In addition, due to the limitations of the intranasal cavity, only small volumes can be administered.

### SUMMARY

Exemplary systems and methods of delivering DNA vaccines are disclosed herein. An exemplary methodology of delivering DNA vaccines includes providing a plasma generator for applying plasma to a treatment area, such as, for example, on skin, tissue or tumor, for a sufficient period of time to open one or more pores, in for example, the skin, tissue or tumor. Applying a topical DNA vaccine to the treatment area and waiting for a period of time to allow the DNA vaccine to travel through the one or more pores. The exemplary methodology further includes applying plasma to the same treatment area at a setting sufficient to promote cellular uptake of the DNA vaccine.

An exemplary noninvasive DNA vaccinating system includes a topical DNA vaccine for applying to a surface, on for example, skin, tissue or tumor and a plasma generator. The plasma generator provides a first plasma treatment to the surface, on for example, skin, tissue or tumor to open one or more pores and applies a second plasma treatment to the same surface to cause cellular uptake of the DNA vaccine into one or more cells.

Another exemplary methodology of vaccinating a body with a DNA vaccine includes applying microsecond or nanosecond pulsed plasma or nanosecond pulsed corona using first parameter set to a treatment area and topically applying the DNA vaccine to the treatment area. The method further includes allowing the plasmid DNA to move through the pores created by the first plasma treatment and then applying microsecond or nanosecond pulsed DBD plasma or nanosecond pulsed corona to the surface at a power setting sufficient to cause cellular uptake of the topically applied plasmid DNA.

Another exemplary methodology of vaccinating a body with a DNA vaccine includes applying microsecond or nanosecond pulsed DBD plasma or nanosecond pulsed corona using a parameter set to a treatment area on skin, tissue or tumor and topically applying the DNA vaccine to the treatment area on skin, tissue or tumor. The parameter set used in this method is sufficient for first allowing the plasmid DNA to move through the pores created in skin, tissue and tumor and simultaneously being up taken by the cells of skin, tissue or tumor via the pores created by plasma treatment in cells.

Another exemplary methodology of vaccinating a body with a DNA vaccine includes intradermally injecting the DNA in to the skin between the epidermis and dermis and topically generating non-thermal DBD plasma on the site of the injection using microsecond or nanosecond pulsed high voltage power supply or generating pulsed corona on the site of the injection using nanosecond pulsed high voltage power supply. Plasma treatment leads to creation of one or more pores in the cells, which enables intracellular uptake of the injected DNA.

### BRIEF DESCRIPTION OF THE DRAWINGS

These and other features and advantages of the present invention will become better understood with regard to the following description and accompanying drawings in which:

FIG. 1 is an exemplary illustration of the layers of skin;

FIG. 2 illustrates an exemplary delivery system for moving molecules through skin, tissue or tumor;

FIG. 3 illustrates another exemplary delivery system for moving molecules across the skin, tissue or tumor

FIG. 4 illustrates a third exemplary delivery system for moving molecules across the skin, tissue or tumor;

FIG. 5 is yet another exemplary delivery system for moving molecules across the skin, tissue or tumor;

FIG. 6 is a plan view of the electrodes of FIG. 5;

FIG. 7 is a schematic diagram of an exemplary methodology of transdermal delivery of DNA vaccines using plasma;

FIG. 8 illustrates another exemplary delivery system for moving molecules across the skin, tissue or tumor; and

FIG. 9 is a cross-section of an exemplary embodiment of an apparatus for treating a surface with plasma to open pores and applying a DNA vaccine to the treated area.

### DETAILED DESCRIPTION

Applicants have developed techniques for moving molecules including DNA across layers of the skin, both intercellularly (between the cells) and intracellularly (into the cells) using cold plasma. Applicants filed U.S. patent application Ser. No. 14/500,144 entitled Method and Apparatus for Delivery of Molecules across Layers of the Tissue on Sep. 29, 2014, which is incorporated herein by reference in its entirety. Some of applicants' exemplary methods utilize plasma for providing a safe, contactless transdermal delivery and cellular uptake of DNA vaccines, which may be referred to herein as plasmaporation.

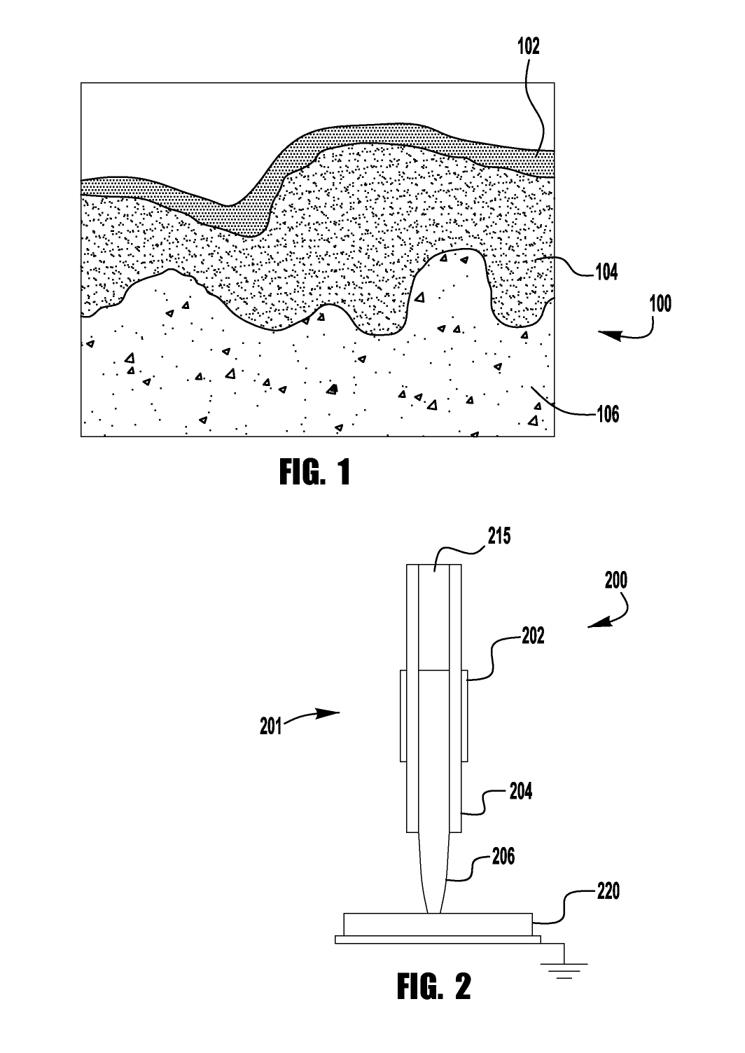

Transdermal delivery requires molecules to pass through the skin. FIG. 1 illustrates the layers of the skin 100. The outer layer of the skin 100 is the stratum corneum (“SC”) 102. The SC 102 is composed of dead, flattened, keratin-rich cells, the corneocytes. These dense cells are surrounded by a complex mixture of intercellular lipids—namely, ceramides, free fatty acids, cholesterol and cholesterol sulfate. The predominant diffusional path for a molecule crossing the SC appears to be intercellular. The remaining layers of the skin are the epidermis (viable epidermis) 104, and the dermis 106

Only a small percentage of compounds can be delivered transdermally because skin 100 has significant barrier properties, namely the highly lipophilic SC 102, that prevents molecules from penetrating or diffusing across the skin. As a result, only relatively small molecules with a molecular weight (MW) of less than 500 Dalton can be administered percutaneously. When topical dermatological therapy, percutaneous systemic therapy or vaccination is the objective, the development of innovative compounds for pharmaceutical applications is restricted to a MW of less than 500 Dalton. In addition, transport of most drugs across the skin is very slow, and lag times to reach steady-state fluxes are measured in hours. Achievement of a therapeutically effective drug level is therefore difficult without artificially enhancing skin permeation using harsh chemical permeation enhancers.

Plasmaporation uses non-thermal plasma or cold plasma, the fourth state of matter for transdermal delivery of DNA vaccines into skin and immune cells. Non-thermal plasma is a partially ionized gas generated at atmospheric pressure using ambient air or other gases and electricity. It is generated by the breakdown of air or other gases present between two electrodes, where often one of them is insulated, under the application of sufficiently high voltage. The second electrode can oftentimes be a dielectric material like living skin, tissue or tumor. A pulsed electric field is used to generate the plasma and to open up temporary (reversible) pores in the skin, tissue or tumor and in cell membranes to promote transdermal delivery and cellular uptake of macromolecules. In some embodiments, the temporary pores remain open for about 1 to about 5 minutes. Electrical parameters used to generate non-thermal plasma can be controlled to achieve reversible plasmaporation. The electrodes do not contact the skin, no needles are required, and contact with non-thermal plasma is painless and safe. In configurations where one or more electrodes are insulated, non-thermal plasma is described as a dielectric barrier discharge (“DBD”) plasma, which is safe and painless even if the insulated electrode contacts the skin, tissue or tumor.

The plasmaporation techniques described herein are an efficient and rapid means of DNA vaccine delivery in a painless and noninvasive manner. In some embodiments, the technique does not require injection of the DNA into the skin. Increased efficiency of delivery of DNA results in less DNA required per immunization, an increased number of doses per unit of DNA, greater ease and more rapid production of the DNA vaccines, smaller production facilities, faster response to a need for new vaccines and lower costs. Large-scale immunization programs can be developed anywhere there is electricity with minimal concern for disposal of biohazardous waste or sharps (syringe needles).

The plasmaporation techniques described herein promote efficient cellular uptake and function of topically applied DNA vaccines into living skin. The exemplary experiments were conducted in a porcine in vivo study because porcine skin resembles human skin in form and function very closely. In the experiments described below, the intracellular uptake of plasmid DNA into skin cells is indicated by the expression of proteins from genes of the plasmid DNA encoding a marker green fluorescent protein (GFP). In some embodiments, the source and preparation of GFP encoding plasmid includes pEGFP-N1 basic vector that encodes a very stable form of AcGFP1 (Aequorea coerulescens GFP) green fluorescent protein (Clontech Laboratories). Plasmid DNA is amplified in E. coli and purified with a commercially available kit (Qiagen Inc.). The plasmid DNA is diluted in sterile phosphate buffered saline at the indicated concentration. In some embodiments, the plasmid DNA encoding the green fluorescence protein was obtained commercially from Aldevron, Inc., Fargo, N. Dak. The plasmid DNA in this case was dissolved and administered in DNAse/RNAse free ultrapure water. Working concentration of the plasmid DNA was 2 mg/ml and 100 μl volume was either applied topically or injected intradermally. No signal is obtained unless intact DNA is taken up intracellularly by the cells followed by expression of the encoded protein.

These experiments demonstrate that plasmaporation is a safe and painless alternative to electroporation and other means of transdermal delivery of DNA. Electrical parameters for generating non-thermal plasma, electrode geometry, DNA concentration, plasmid DNA construction and the mode of plasmid DNA application to the skin (injected or topically applied on the skin surface) can be varied to optimize the process. In addition to those uses described above and many other uses, plasmaporation of DNA vaccines may also be used to prevent viral infections (e.g. herpes simplex virus, Ebola, etc.) and treat cancers (e.g. Her-2/neu Breast cancer), which are vaccine targets currently under investigation.

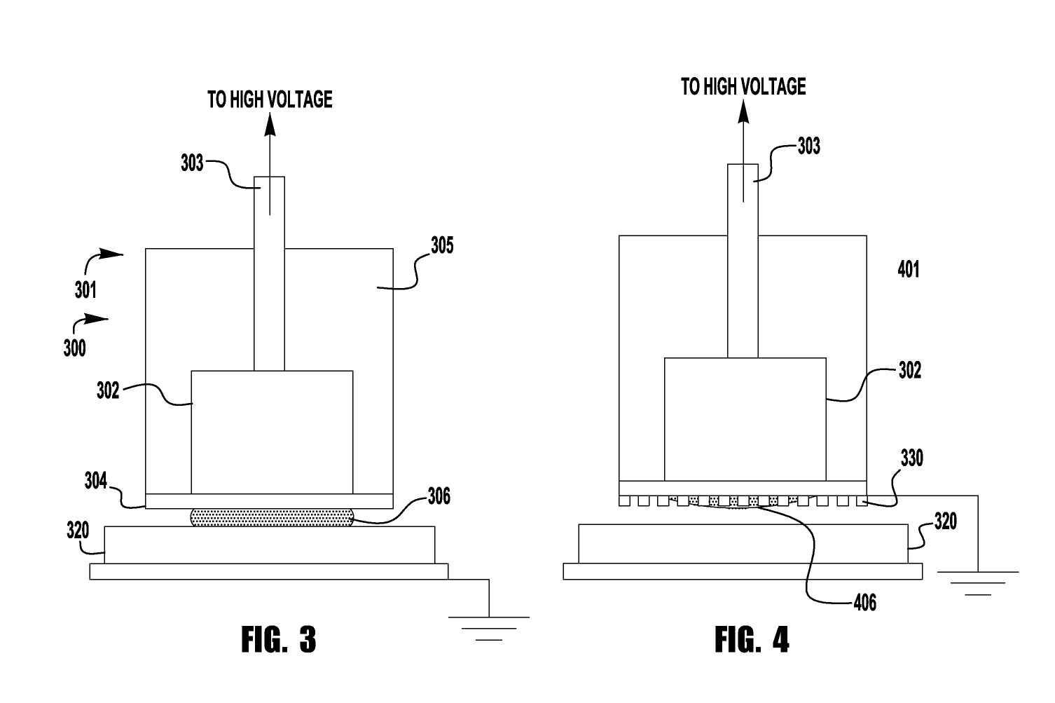

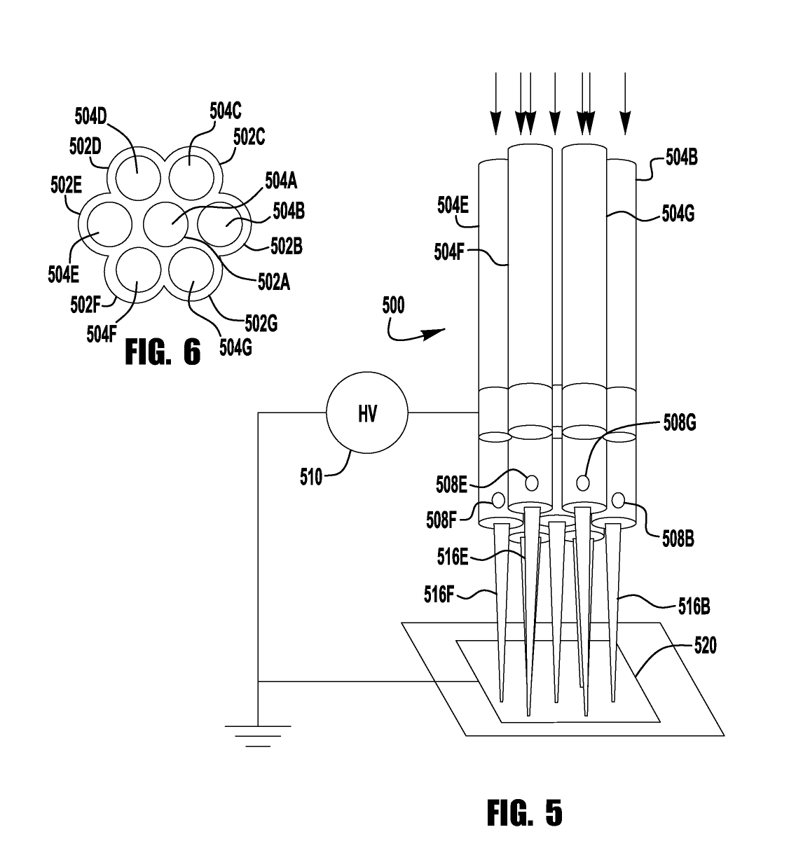

In some exemplary embodiments, plasmaporation involves the use of planar DBD plasma generators (FIGS. 3 and 4) or DBD jet plasma generators (FIGS. 2 and 5) or pulsed corona generators (FIG. 8) for needle-free transdermal delivery of macromolecules. Depending on the parameters used to generate plasma (time of treatment, applied voltage, pulse repetition frequency, pulse duration, number of applied pulses, duty cycle etc.), the depth of permeation of the macromolecules can be regulated to ensure delivery to the target layer, which may be, for example, the epidermis or the dermis in skin. As such, non-thermal plasmaporation techniques described herein can promote both rapid transdermal delivery and enhance efficient cellular uptake of a DNA vaccines delivered at atmospheric pressure and room temperature without the need for disposable electrodes or needles, as needed for electroporation and other prior techniques.

Applicants have previously demonstrated that plasmaporation can enhance transdermal delivery of topically applied dextran molecules with molecular weights up to 70 kDa, proteins up to 115 kDa, and nanoparticles up to 50 nm in diameter across ex vivo porcine skin within 15 minutes and without causing any skin damage, as described in U.S. Non-Provisional application Ser. No. 14/500,144 entitled Method and Apparatus for Delivery of Molecules Across Layers of Tissue on Sep. 29, 2014, which is incorporated herein by reference.

Atmospheric pressure non-thermal plasmas can drive macromolecules through the surface and into ex vivo porcine skin without harming the skin in any way. Non-thermal plasma enabled skin poration provides a non-invasive and safe means for transdermal delivery and cellular uptake of DNA vaccines at room temperature and atmospheric pressure without the possible pain, muscle contractions and other side effects associated with electroporation. As the application of the method does not require disposable electrodes or needles, the need for disposal of biohazardous waste and illicit reuse of biohazardous consumables is eliminated. An additional benefit of using non-thermal plasma is that the generated reactive species can sterilize the skin during plasmaporation.

In the plasma phase, neutral gas atoms (or molecules), electrons, positive/negative ions, and highly energetic radicals are generated. Their generation and concentration depend, in part, on the physical and chemical properties of the gas being used to generate the plasma, the device design as well as the electrical parameters used to generate the plasma. The strength of the electric field generated by non-thermal plasma on skin can be tuned by varying the time of plasma treatment; gap between the electrode and the skin, tissue or tumor; applied voltage; pulse duration; number of applied pulses, pulse repetition frequency and duty cycle to localize delivery. These parameters allow control of the depth and delivery amount of macromolecules across the skin, tissue or tumor allowing treatment of the targeted skin or tissue layer with the optimal dose, potentially minimizing the amount of expensive DNA vaccine for each treatment.

Plasma operating parameters may vary and for microsecond pulsed plasma applications, power supply settings (for both the topical applications and the injected applications) may include setups ranging in a pulse repetition frequency from 50-3500 Hz with a pulse duration of between about 1-10 μs at a voltage of between about 11-20 kV with a duty cycle of between about 1-100% and voltage rise times of between about 1-5 V/ns. Treatment times may range from between about 5 seconds and about 180 seconds. Similarly, for the nanosecond pulsed plasma applications (for both the topical and injected experiments) power settings may be set at a pulse repetition frequency of between about 2-20000 Hz at pulse durations of between about 1 ns-500 ns and voltages of between about 3-20 kV at voltage rise times of 0.5-10 kV/ns for the continuous applications. For discrete pulse applications, the settings may be between about 1-100 pulses with pulse durations of about 1 ns-500 ns with voltages of between about 3-20 kV at voltage rise times of 0.5-10 kV/ns. Treatment times may be between about 1 seconds and about 300 seconds. The pulses may have positive or negative polarity. Additionally, for the nanosecond pulsed corona application power settings may be set at a pulse repetition frequency of between about 1-1000 Hz at pulse duration of between about 1 ns-60 ns and voltage of between about 3-20 kV at voltage rise times of 0.5-10 kV/ns or the continuous application. For discrete pulse applications of corona, the settings may be between about 1-100 pulses with pulse duration of about 1 ns-60 ns with voltages of between about 3-20 kV at voltage rise times of 0.5-10 kV/ns.

In some embodiments, a helium DBD jet with optimal parameters to achieve safe (without thermal or other damage to the skin) cellular DNA uptake and robust expression of encoded GFP is used. The device produces a focused plasma beam on the skin at a distance of 5-50 mm away from the tip of the electrode allowing a more remote application. FIG. 2 illustrates an exemplary embodiment of a delivery system 200 for delivering DNA plasmids through skin 220. The exemplary delivery system 200 includes a non-thermal plasma generator 201 that includes a high voltage tubular metal electrode 202 and a borosilicate glass tube 204 serving as the dielectric. Plasma generator 201 is a floating-electrode dielectric barrier discharge (DBD) plasma generator that generates a plasma “jet” 206.

Plasma generator 201 includes a gas feed 215. Exemplary gases that may be used to feed the plasma jet include He, He+O₂, N₂, He+N₂, Ar, Ar+O₂, Ar+N₂, and the like. Gases resulting from the evaporation of liquid solutions can also be used. Examples of vaporized liquids may include water, ethanol, organic solvents and the like. These vaporized liquids may be mixed with additive compounds of pharmaceutical substances, permeation enhancers, etc. The evaporated liquids and additives may be used with the gases identified above in various concentrations or without the gases.

Plasma generator 201 includes a power supply, not shown. The power supply is a high voltage supply and may have a number of different wave forms, such as, for example, a constant, ramp-up, ramp-down, pulsed, nanosecond pulsed, microsecond pulsed, square, sinusoidal, decaying sinusoidal, random, in-phase, out-of-phase, and the like. In some exemplary embodiments, the power supply was a microsecond pulsed power supply. In some exemplary embodiments, the power supply was a nanosecond pulsed power supply. In some exemplary embodiments, the plasma 206 was generated by applying alternating polarity pulsed voltage. In some embodiments, the voltage had a pulse width of between about 1-10 μs (pulse repetition frequency: 50 Hz to 3.5 kHz) with a rise time of 5V/ns and a magnitude of about ˜20 kV (peak-to-peak) at a power density of 0.1-10 W/cm². During operation, the plasma jet 206 is in direct contact with the skin 220.

The plasma allows the electric field to reach the skin and deposit electrical charges to develop a voltage potential across the skin, which leads to intracellular and intercellular poration. Plasmaporation, described above is non-invasive as the plasma electrode is not in contact with the tissue or substrate to be treated.

With respect to intracellular poration, the transmembrane voltage of fluid lipid bilayer membranes needs to reach at least about 0.2 V. The transmembrane voltage charges the lipid bilayer membranes, causes rapid, localized structural rearrangements within the membrane and causes transitions to water-filled membrane structures, which perforate the membrane forming “aqueous pathways” or “pores.” The aqueous pathways or pores allow an overall increase in ionic and molecular transport. The transmembrane voltage is believed to create primary membrane “pores” with a minimum radius of about approximately 1 nm. In addition, the applied electric field results in rapid changes in the state of polarization that deform mechanically unconstrained cell membranes (e.g., suspended vesicles and cells) and cause ionic charge redistribution governed by electrolyte conductivities.

The electrical pulses used to generate the plasma jet 206 also cause intercellular poration. The SC, which is about 15-25 μm thick, is the most electrically resistive part of skin. The application of electrical pulses used to generate the plasma jet 206 gives rise to a transdermal voltage ranging between about 50V and about 100V, which causes poration of the multilamellar bilayers within the SC. At these levels of applied transdermal voltage, poration of cell linings of sweat ducts and hair follicles could also occur.

Upon removal of the plasma source from the treated area the pores tend to close again and thus, the process is reversible. Some pores remain open for an extended period of time, during which molecules can continue to cross the cell membrane via diffusion. In some other cases, the applied transmembrane potential might exceed a threshold value so that the formed pores remain open indefinitely. This process is called irreversible poration and can be beneficial for cancer treatment.

When electric pulses are applied to the skin, the absorbed energy can cause localized heating and damage to the skin. Energy greater than 100 J/cm² deposited on intact skin results in second degree burns and thermal damage to the underlying intact skin. One method of overcoming this problem is to apply short duration pulses repetitively, which allows the same amount of energy that would otherwise cause damage to be transferred without causing localized heating and skin damage. In some embodiments, the energy deposited on intact skin is less than about 50 J/cm², in some embodiments, the energy deposited on intact skin is less than about 25 J/cm², in some embodiments, the energy deposited on intact skin is less than about 10 J/cm², in some embodiments, the energy deposited on intact skin is less than about 5 J/cm², and in some embodiments, the energy deposited on intact skin is less than about 3 J/cm². However, when treating wounds, the energy may be increased, to for example, 500 J/cm², without causing burns. In some embodiments, energy in the range of 500 J/cm² may be used to coagulate blood.

In addition, damage to the skin may occur from localized plasma micro-discharges, also known as “streamers,” that occur with non-uniform electric fields and also due to non-uniformity of the surface being treated (like skin, tissue or tumor). This problem may be overcome by creating a uniform electric field. In some embodiments, helium gas may be used as the gas supplied to plasma generator 201. It has been discovered, that use of helium provides a uniform plasma field and minimizes streamers. In addition, a nanosecond pulsed power supply provides a more uniform plasma field and accordingly potential damage to skin. Also, skin damage can be avoided by reducing the power level, time of treatment, frequency, duty-cycle and pulse duration of the power supply and by increasing the spacing between the plasma electrode and skin, tissue or tumor to be treated.

After the application of plasma to cause plasmaporation and once the plasma generating device 206 is turned off, the multilamellar system of aqueous pathways remain open for a period of time that may be up to about a few minutes to few hours.

Other types of plasma generators may be used for delivery systems, such as, for example, nanosecond pulsed DBD plasma, microsecond pulsed DBD plasma, sinusoidal DBD plasma, resistive barrier discharge plasma, surface DBD plasma, 2-D or 3-D array of nanosecond pulsed corona, 2-D or 3-D array of DBD plasma jets operating under a continuous mode or under a controlled duty cycle ranging from 1-100% and the like. Not all plasma generators may be used to successfully induce poration. Plasma generators that deliver high electric current or that significantly increase the temperature of the object being treated are not suitable for plasmaporation, including thermal plasma, gliding arc discharges, plasmatrons, etc. Such plasma generators may cause electric shock, severe thermal damage, muscle contraction and pain or do not deliver sufficient charges to the substrate being treated, which would mean no or very weak applied electric field and hence no induced poration.

Suitable plasma generators have dominating currents that are displacement currents at low power and/or high frequencies. Displacement current has units of electric current density, and an associated magnetic field just as conduction current has, however, it is not an electric current of moving charges, but rather a time-varying electric field. The electric field is applied to the skin by an insulated electrode that is not in contact with the skin. Because the electrode is insulated and is not in contact with the skin, there is minimal flow of conduction current into the skin. Strong conduction currents would cause electric shock, thermal damage, muscle contraction and pain that is associated with electroporation.

For larger treatment areas, electrode configurations consisting of multiple plasma jets or larger area flat electrodes (not shown) may be used. In the case of more complex 3D surfaces, a controlled plasma module (not shown) may move around a stationary target or the surface to be exposed to the plasma may be placed on a movable stage. In some embodiments, one or more plasma jets or can be attached to a robotic arm that is programmed to move in a manner that exposes one or more target areas to a plasma plume or jet.

In addition, in some embodiments, the plasma generator 201 may be coupled with a biomolecule/drug delivery system, where molecules may be transported to the treatment area through needle-free injection, application of pressurized gas, evaporation, spraying and or misting. In some embodiments, this may assist with the pretreatment of the surface.

In some embodiments where it is essential to reduce the plasma temperature and enhance skin permeation following plasmaporation, it is beneficial to generate non-thermal plasma using He, Ar, Ne, Xe and the like, air, or mixtures of inert gases with small percentage (0.1%-20%) of other gases such as O₂ and N₂ and mixtures of inert gases with vaporized liquids including water, dimethyl sulfoxide (DMSO), ethanol, isopropyl alcohol, n-butanol, with or without additives and the like.

In some embodiments, a non-thermal planar DBD plasma generator is used to promote transdermal delivery as well as cellular uptake of plasmid DNA topically applied on the surface of porcine skin. FIG. 3 illustrates an exemplary non-thermal planar DBD delivery system 300. Delivery system 300 includes a plasma generator 301. Plasma generator 301 includes a high voltage wire 303 connected to an electrode 302 on a first end and a high voltage power supply (not shown) on the second end. Suitable high voltage supplies are described above. A dielectric barrier 304 is located below the high voltage electrode 302. In addition, the high voltage electrode 302 is located within a housing 305. Plasma generator 301 is a non-thermal dielectric barrier discharge (DBD) generator. Plasma 306 is generated by the plasma generator 301. FIG. 3 also includes skin 320. For the exemplary experimental results disclosed herein, skin 320 is live porcine skin. Cold plasma is generated directly in contact with the skin when the DBD plasma source is placed at a distance of 1-5 mm from the surface of skin.

Direct plasma 306 was generated by applying alternating polarity pulsed voltage to the electrode 302. In some embodiments, the applied voltage may have a pulse width of between about 1-10 μs (pulse repetition frequency: 50 Hz to 30 kHz) with a magnitude of about ˜20 kV (peak-to-peak) and a voltage rise time of about 1-10 V/ns. The power supply (not shown) may be a variable voltage and variable frequency power supply. A 1 mm thick clear quartz slide, alumina or Teflon may be used as the insulating dielectric barrier 304 and to cover the electrode 302. Electrode 302 may be a 2.54 cm diameter copper, brass or other conductive material. The discharge gap between the dielectric barrier 304 and the porcine skin 320 may be about 4 mm±1 mm. In some embodiments, the pulse waveform may have an amplitude of about 22 kV (peak-to-peak), a duration of about 9 μs, with rise time of about 5 V/ns. The discharge power density may be between about 0.1 W/cm² to 2.08 W/cm². The plasma treatment dose in J/cm² may be calculated by multiplying the plasma discharge power density by the plasma treatment duration.

In addition, indirect plasma 406 may be created with a plasma generator 401. Plasma generator 401 is similar to plasma generator 301, except that plasma generator 401 includes a metal mesh 330 that filters the plasma 406. The metal mesh 300 prevents charged ions and electrons from passing through, but allows the neutral species to pass through and contact the skin. The neutral species may be referred to as “afterglow.”

FIG. 5 is a schematic of yet another exemplary embodiment of a delivery system 500. FIG. 6 is a plan view of the electrodes of delivery system 500. Delivery system 500 includes a plurality of DBD jets. The exemplary delivery system 500 has an array of DBD jets in a honeycomb shape; however, many other configurations may be used such as, linear, triangular, square, pentagonal, hexagonal, octagonal, etc.

The DBD jets have glass tubes 504A, 504B, 504C, 504D, 504E, 504F and 504G. A metal electrode 502 includes a plurality of cylindrical openings 502A, 502B, 502C, 502D, 502E, 502F, and 502G that receive each of the corresponding glass tubes 504A, 504B, 504C, 504D, 504E, 504F, and 504G. Optionally, multiple metal electrodes may be used. The metal electrode 502 may have an insulating covering (not shown) to prevent shock. The metal electrode 502 is connected to a high voltage source as described above.

The DBD jets have a gas flow inlet located at a first end and have a plasma jet 516A, 516B, 516C, 516D, 516E, 516F and 516G out the other. As described above, the gas may be, for example, He, Ar, Ne, Xe, air, He+Air, Ar+Air, Ne+Air, Xe+Air, or the like. In addition, each glass tube 504A, 504B, 504C, 504D, 504E, 504F and 504G has an inlet 508A, 508B, 508C, 508D, 508E, 508F, and 508G located along the glass tube for receiving vaporized liquid additives. These inlets may be located above or below electrode 502. The exemplary transdermal delivery system 500 utilizes skin, tissue or tumor as a ground electrode.

In the exemplary embodiment of FIG. 2, the skin, tissue or tumor 220 is directly exposed to the plasma 206 containing neutral and charged species. Similarly, in the exemplary embodiment of FIG. 3, with direct plasma generator 301, the electrical discharge occurred between the dielectric barrier 304 and the skin 320, which exposed the skin directly to neutral reactive species and charged particles.

Indirect plasma created by plasma generator 401 utilized a grounded copper mesh (16×16 mesh size with a 0.011″ wire diameter and a 0.052″ opening size) that was placed between the high voltage electrode and the skin, which eliminated charged particles from contacting the exposed surface of the skin.

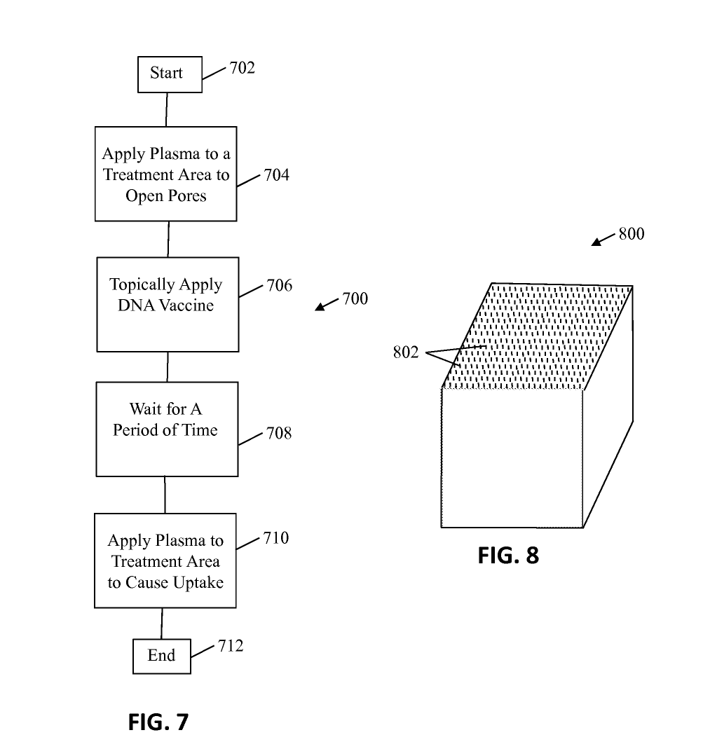

FIG. 7 is an exemplary methodology 700 for transdermal delivery of DNA vaccines. The exemplary methodology 700 begins at block 702. At block 704, plasma is applied to a treatment area to open one or more pores in skin, tissue or tumor. At block 706 a DNA vaccine is topically applied to the target area on skin, tissue or tumor and a waiting period occurs at block 708. In some embodiments, the waiting period is about 3 hours or less, in some embodiments the waiting period is about 2 hours or less. In some embodiments, the waiting period is about an hour or less, and in some embodiments is about 30 minutes or less. A second plasma application occurs at block 710 to porate cells in skin, tissue or tumor leading to the enhancement of intracellular uptake of the DNA vaccine and the exemplary methodology ends at block 712.

FIG. 8 is yet another exemplary embodiment 800 of a plasma system for enabling safe and efficient transdermal delivery of topically applied plasmid DNA and promoting the intracellular uptake of the DNA by cells of skin, tissue or tumor. This exemplary embodiment 800 utilizes a pulsed corona array generated by a nanosecond pulsed power supply (not shown). The pulsed corona array is made up of a plurality of sharp tips 802, which in this exemplary embodiment are stainless steel machined tips 0.1016 mm thick and 1 mm apart from each other. The sharp tips 802 form a 2D array of pulsed corona electrodes.

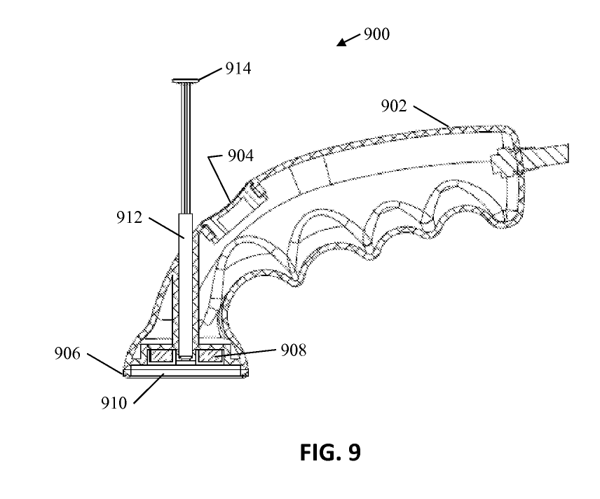

FIG. 9 is a cross-section of an exemplary embodiment of a vaccinating apparatus 900 for treating a surface with plasma to open pores in the surface and/or cells and applying a DNA vaccine to the treated area. Vaccinating apparatus 900 includes a housing 902, an on-off switch 904, surface contact ring 906, high voltage electrode 908, treatment chamber 910, DNA vaccine chamber 912 and plunder 912. In addition, vaccinating apparatus 900 includes a power supply (not shown) that may provide the required power including all of the power settings identified herein. In some embodiments surface contact ring 906 is detachable and may be replaced after each use. In addition, in some embodiments surface contact ring 906 includes a grounding ring (not shown).

Vaccinating apparatus 900 may be operated in several different modes. In one exemplary embodiment, power settings are selected on the power supply (not shown) and an operator places the surface contact ring 906 on a surface, such as, for example, a person's skin, tissue, tumor, or the like. The operator presses the start button 904 causing the desired voltage(s) to be applied to high voltage electrode 908 creating plasma in treatment chamber 910. After a sufficient period, the plasma applied to the surface of the skin to open pores in the skin. The operator presses plunger 914 downward causing a DNA vaccine, which is stored in vaccine chamber 912, to be injected into treatment chamber 910. After a sufficient period of time has elapsed to allow the DNA vaccine to pass through the opened pores in the skin, tissue, tumor or the like, a desired power is applied to the high voltage electrode to create plasma to open pores in one or more cells to cause cellular uptake of the DNA vaccine.

In another exemplary embodiment, power settings are selected on the power supply (not shown) and an operator places surface contact ring 906 on a surface, such as, for example, a person's skin, tissue, tumor, or the like. The operator presses the start button 904 causing a selected voltage(s) to be applied to high voltage electrode 908 creating plasma in treatment chamber 910. The plasma is applied to the surface to open pores in the skin, tissue, tumor, or the like and to open pores in the cells. The operator presses plunger 914 downward causing the DNA vaccine, which is stored in vaccine chamber 912, to be injected into treatment chamber 910. After a sufficient period of time has elapsed to allow the DNA vaccine to pass through the opened pores and is taken up by the cells.

### Experimental Results

A number of experiments were conducted on live animals. Three 5-7 month old Yucatan minipigs were utilized in three live animal experiments. Each study included a number of experiments on a single minipig. Experimental controls included: no plasma treatment, no plasmid DNA application; no plasma treatment with plasmid DNA injected intradermally; and plasmid DNA injected intradermally followed by electroporation (current state of the art). Experimental samples included: microsecond pulsed plasma after intradermal injection; nanosecond pulsed plasma after intradermal injection; microsecond pulsed plasma followed by topical plasmid DNA application; and nanosecond pulsed plasma followed by topical plasmid DNA application.

The first control involved no plasmid DNA application and consequently no resulting expression of green fluorescent protein (GFP) was observed 2 days after the animal was treated.

In the second control, plasmid DNA was injected intradermally (“ID”), which resulted in some expression of GFP. The expression of GFP was, however, mostly localized in the dermis with very little expression in the epidermis (Expression in the epidermis important in obtaining a robust immune response to the administered vaccine). A normalized intensity was derived based on the data obtained from the biopsies. The normalized intensities are shown in Chart I below.

CHART I

| | Injected Control | Electroporation | Plasma |
|---|---|---|---|
| Minipig | (Normalized Intensity) | (Normalized Intensity) | Treatment |
| #1 | 8.57E+06 | | |
| #2 | 1.95E+06 | | |
| #3 | 3.96E+06 | | |

In the third control, intradermal injection of plasmid DNA was followed by electroporation using a standard two-needle electrode at an electric field of 200 V/cm, 16 pulses 150 ms in duration at a frequency of 1 Hz with 90° rotation of the electrode after 8 pulses. After electroporation, there was strong expression of GFP observed in the epidermis and little expression of GFP observed in the dermis. A normalized intensity was derived based on the data obtained from the biopsies. The normalized intensities are shown below in Chart II. As described above, electroporation has numerous side effects including pain. In addition, electroporation requires an intradermal injection of the plasmid DNA prior to treatment because unlike plasma treatment it cannot be used to promote intercellular delivery of topically applied plasmid DNA across the highly resistive stratum corneum or tissue or tumor.

CHART II

| | Injected Control | Electroporation | Plasma |
|---|---|---|---|
| Minipig | Normalized Intensity | Normalized Intensity | Treatment |
| #1 | 8.57E+06 | 1.78E+07 | |
| #2 | 1.95E+06 | 4.13E+06 | |
| #3 | 3.96E+06 | 4.29E+06 | |

In a first set of experiments, plasmid DNA (100 μl of 2 mg/ml) was injected intradermally and the injection was followed by plasma treatment using a microsecond pulsed DBD plasma with a power supply set at 3500 Hz, a pulse duration of 5 μs, a voltage of 15 kV and a 100% duty cycle. The area was treated for the times indicated in the chart below. Two days after DBD plasma treatment, robust expression of the protein encoded by the plasmid was observed in both the epidermis and the dermis. A normalized intensity was derived based on the data obtained from the biopsies. The normalized intensities are listed in Chart III below.

CHART III

| | Injected | Electro- | |
|---|---|---|---|
| | Control | poration | |
| | Normalized | Normalized | Microsecond |
| Minipig | Intensity | Intensity | pulsed DBD Treatment |
| #1 | 8.57E+6¹ | 1.78E+07 | 2.03E+07 for two 30 s treatments |
| | | | 1.42E+07 for two 60 s treatment |
| #2 | 1.95E+06 | 4.13E+06 | 5.01E+06 for one 30 s treatment |
| #3 | 3.96E+06 | 4.29E+06 | 7.98E+06 for one 45 s treatment |

¹The intensity of signal depends on a number of factors, such as the thickness of the skin slice, histological handling, quality of DNA, etc. While the intensity is consistent across each experiment, the intensity may not correlate precisely across experiments.

The data demonstrates that use of microsecond continuous DBD plasma treatment resulted in 137%, 66%, 166% and 102%, respectively, increase in cellular uptake over the injected control. In addition, the data demonstrates that use of microsecond continuous DBD plasma is superior to electroporation for causing cellular uptake. The data demonstrates that use of microsecond continuous DBD treatment resulted in 14%. 20%, 21% and 86% increase, respectively, in cellular uptake and GFP expression over electroporation.

In a second set of experiments, the skin was treated with microsecond pulsed DBD plasma. The power source was set at 3500 Hz with pulse duration of 5 μs and a voltage of 15 kV. The treatment time was for 120 seconds. A 100 μl plasmid DNA solution was applied for a period of time (“hold time”) identified in the chart below to the area treated with plasma. A second DBD plasma treatment with the power source set at 3500 Hz with pulse duration of 5 μs at a voltage of 15 kV was applied for 60 seconds.

Robust expression of the GFP was observed in both the epidermis and the dermis. These experiments demonstrated that plasmaporation is able to achieve intercellular delivery of plasmid DNA to the correct layer of the skin and was able to achieve intracellular uptake of the plasmid DNA by cells in the epidermis and dermis. In addition, this exemplary methodology eliminates the need for needle sticks, associated disposal of biohazardous waste and illicit reuse of sharps waste. The normalized intensities are listed in Chart IV below.

CHART IV

| | | Electroporation | |
|---|---|---|---|
| | Injected Control | Normalized | Microsecond |
| Minipig | Normalized Intensity | Intensity | pulsed DBD Treatment |
| #1 | 8.5736E+6 | 1.7801E+07 | 9.4004E+06 for |
| | | | two 35 min hold times |
| | | | 2.1710E+07 for |
| | | | two 60 min hold times |
| #2 | 1.956E+06 | 4.134E+06 | 3.341E+06 for |
| | | | one 60 min hold time² |
| #3 | 3.960E+06 | 4.295E+06 | 4.399E+06 for |
| | | | one 15 min hold time³ |

²No stratum corneum was present in two histologically processed samples as this skin was extremely sensitive due to a previous injury to the animal, and the results were not included in the data.
³There was a result for a 60 minute hold time that resulted in no expression and was not included in the results and this was attributed to variation from animal to animal.

The data demonstrates that treating the skin with microsecond continuous DBD plasma, topically applying plasmid DNA and use of a second microsecond continuous DBD plasma treatment to cause cellular uptake resulted in 10%, 153%, 71% and 11%, respectively, improvement in cellular uptake over the injected control. Some of the data demonstrated that this topical application methodology was as good as or better than electroporation without the pain and other side effects associated with electroporation. However, some experimental results indicate this methodology may need to be modified and/or the parameters may need to be adjusted. The results also indicate that it may be advantageous to combine one or more of the plasma treatments, such as, for example, using the microsecond continuous DBD plasma treatment to treat the skin and open the pores and using a nanosecond pulsed treatment (disclosed below) to cause intracellular uptake.

In a third set of experiments, plasmid DNA (100 μl of 2 mg/ml) was injected intradermally and followed by a number of pulses of DBD plasma applied using a nanosecond pulsed power supply set at 20 kV with a pulse duration of 500 ns. Robust expression of the GFP was observed in both the epidermis and the dermis 2 days after the plasma treatment. The experiments demonstrated that in some embodiments, only 25 pulses are needed, and therefore this exemplary methodology may be extremely fast, safe, and plasma may be applied for a very short duration. In addition, this methodology uses less power and could be enabled using a hand-held battery powered plasma applicator. The normalized intensities are listed in Chart V below.

CHART V

| | Injected Control | Electroporation | |
|---|---|---|---|
| | Normalized | Normalized | Nanosecond pulsed |
| Minipig | Intensity | Intensity | DBD Treatment |
| #1 | 8.57E+06 | 1.78E+07 | 2.08E+07 for 25 pulses |
| #2 | 1.95E+06 | 4.13E+06 | 5.28E+06 for 25 pulses |
| | | | 5.30E+06 for 100 pulses |
| | | | 3.40E+06 for 75 pulses |
| #3 | 3.96E+06 | 4.29E+06 | 8.61E+06 for 25 pulses |

The data demonstrates that use of nanosecond pulsed DBD plasma treatment resulted in 143%, 170%, 171%, 74% and 117%, respectively, improvement in cellular uptake over the injected control. In addition, the data demonstrates that use of nanosecond pulsed DBD plasma treatment is superior to electroporation for enhancing cellular uptake. The data demonstrates that use of nanosecond pulsed DBD plasma treatment resulted in 17%, 28%, 28%, −18% and 100% increase respectively improvement over cellular uptake than electroporation.

In a fourth set of experiments, plasmid DNA (100 μl of 2 mg/ml) was injected intradermally and followed by plasma treatment for a number of seconds. The plasma treatment was continuous nanosecond pulsed DBD plasma treatment with a power supply set at 20 kV, 200 Hz and a pulse duration of 200 ns. Robust expression of the GFP was observed in both the epidermis and the dermis 2 days after the plasma treatment with continuous nanosecond DBD plasma. The normalized intensities are listed in Chart VI below.

CHART VI

| | Injected Control | Electroporation | |
|---|---|---|---|
| | Normalized | Normalized | Nanosecond pulsed |
| Minipig | Intensity | Intensity | DBD Treatment |
| #1 | 8.57E+6 | 1.78E+07 | 1.07E+07 for 60 seconds |
| | | | 1.13E+07 for 80 seconds |
| #2 | 1.95E+06 | 4.13E+06 | 4.8E+06 for 120 seconds |
| #3 | 3.96E+06 | 4.29E+06 | 3.8E+06 for 120 seconds |

The data demonstrates that use of continuous nanosecond pulsed DBD plasma treatment resulted in 26%, 33%, 150% and −3.82% respectively, improvement in cellular uptake over the injected control. The results do not indicate that these settings are superior to electroporation.

In a fifth set of experiments, a 120 second continuous nanosecond pulsed DBD plasma treatment with the power supply set at 200 Hz, 20 kV with a pulse duration of 200 ns was applied to the skin. A 100 μl plasmid DNA solution was topically applied to the treated area for either 30 minutes or 60 minutes. A second 120 second continuous nanosecond DBD plasma treatment with the power supply set at 200 Hz, 20 kV with pulse duration of 200 ns was applied to the treatment area. Weak expression of the GFP was observed mostly in the dermis and no expression was observed in the epidermal layer.

In a sixth set of experiments, plasmid DNA (100 μl of 2 mg/ml) was injected intradermally and followed by plasma treatment using pulsed or continuous application using the 2D pulsed corona array. In one instance skin was treated with 25 pulses of 500 ns pulse duration at 20 kV applied voltage and in the second instance skin was treated for 30 s with a frequency of 100 Hz, pulse duration of 80 ns and applied voltage of 20 kV. In both cases robust expression of the GFP was observed in both the epidermis and the dermis 2 days after the plasma treatment with the pulsed corona array. The normalized intensities are listed in Chart VII below.

CHART VII

| | Injected Control | Electroporation | |
|---|---|---|---|
| | Normalized | Normalized | Nanosecond pulsed |
| Minipig | Intensity | Intensity | Corona Treatment |
| #3 | 3.96E+06 | 4.29E+06 | 8.61E+06 for 25 pulses |
| | | | 7.81E+06 for 30 seconds |

The data demonstrates that use of nanosecond pulsed corona treatment resulted in 117% and 97% respectively, improvement in cellular uptake over the injected control. In addition, the data demonstrates that use of nanosecond pulsed corona treatment is superior to electroporation for enhancing cellular uptake. The data demonstrates that use of pulsed nanosecond corona resulted in 100% and 82% increase respectively, improvement over cellular uptake than electroporation. The experiments demonstrated that in some embodiments, only 25 pulses are needed, and therefore this exemplary methodology may be extremely fast, and plasma may be applied for a very short duration. In addition, this methodology uses less power and could be enabled using a hand-held battery powered plasma applicator.

While the present invention has been illustrated by the description of embodiments thereof and while the embodiments have been described in considerable detail, it is not the intention of the applicant to restrict or in any way limit the scope of the appended claims to such detail. Additional advantages and modifications will readily appear to those skilled in the art. For example, Flexible and wearable electrodes may be developed and the generation of the non-thermal plasma can be optimized for transdermal delivery. The methods described herein may be used to cause cellular uptake of other macromolecules (e.g. antibodies, drugs) in addition to DNA vaccines. Therefore, the invention, in its broader aspects, is not limited to the specific details, the representative apparatus and illustrative examples shown and described. Accordingly, departures may be made from such details without departing from the spirit or scope of the applicant's general inventive concept.

We claim:

1. A method of delivering a DNA vaccine comprising:
providing a plasma generator for applying plasma to a treatment area for a sufficient period of time to open one or more pores;
applying a topical DNA vaccine to the treatment area;
waiting for a period of time to allow the topically applied DNA vaccine to travel through the one or more pores;
applying plasma to the treatment area at a setting sufficient to promote cellular uptake of the DNA vaccine.

2. The method of claim 1 wherein the plasma used to open one or more pores is generated at atmospheric pressure using ambient air.

3. The method of claim 1 wherein the plasma used to open one or more pores is one of a microsecond pulsed plasma and a nanosecond pulsed plasma.

4. The method of claim 3 wherein the plasma used to open one or more pores is one of a DBD plasma and a nanosecond pulsed corona array.

5. The method of claim 1 wherein the period of time to allow the DNA vaccine to travel through the one or more pores is less than about 1 hour.

6. The method of claim 1 wherein the period of time to allow the DNA vaccine to travel through the one or more pores is less than about 30 minutes.

7. The method of claim 1 wherein the cellular uptake occurs in epidermal cells.

8. The method of claim 1 wherein the cellular uptake occurs in immune cells residing in the dermis.

9. The method of claim 1 wherein the cellular uptake occurs in cells of tissue.

10. The method of claim 1 wherein the cellular uptake occurs in tumor cells.

11. A noninvasive DNA vaccinating system comprising:
a topical DNA vaccine for applying to the surface of the skin, tissue or tumor; and
a plasma generator for providing a first plasma treatment to the skin, tissue or tumor to open one or more pores in the skin, tissue or tumor and for providing a second plasma treatment to the skin, tissue or tumor to cause cellular uptake of the DNA vaccine into one or more cells of skin, tissue or tumor.

12. The noninvasive DNA vaccinating system of claim 11 wherein the plasma generator is one of a microsecond pulsed DBD plasma generator, a nanosecond pulsed DBD plasma and a nanosecond pulsed corona.

13. A method of vaccinating a body with a DNA vaccine comprising:
treating an area with a pulsed plasma;
applying a DNA vaccine topically on the treated area; and
applying plasma to the surface of the treated area at a power setting sufficient to cause cellular uptake of the plasmid DNA.

14. The method of vaccinating a body of claim 13 further comprising waiting for a set period of time between topically applying the DNA Vaccine and applying plasma to the surface of the treatment area.

15. The method of vaccinating a body of claim 14 wherein the set period of time is less than about 1 hour.

16. The method of vaccinating a body of claim 14 wherein the set period of time is less than about 30 minutes.

17. A method of vaccinating a body with a DNA vaccine comprising:
applying plasma to the surface of skin, tissue or tumor at a power setting sufficient to open pores in the skin, tissue or tumor and create pores in the cells;
applying a DNA vaccine topically on the treated area causing it to move through the pores created in skin, tissue or tumor and be taken up intracellularly via the pores created in the cells of the skin, tissue or tumor.

18. The method of claim 17 wherein the plasma used to open one or more pores is generated at atmospheric pressure using ambient air.

19. The method of claim 17 wherein the plasma used to open one or more pores is one of a microsecond pulsed plasma and a nanosecond pulsed plasma.

20. The method of claim 19 wherein the plasma used to open one or more pores is one of DBD plasma and a pulsed corona plasma.
